(19) United States
(12) Patent Application Publication
(10) Pub. No.: US 2014/0147524 A1
(43) Pub. Date: May 29, 2014

(54) Truely Re'Vived

(71) Applicant: Seanterel Latrice Darby, Waxahachie, TX (US)

(72) Inventor: Seanterel Latrice Darby, Waxahachie, TX (US)

(21) Appl. No.: 13/684,503

(22) Filed: Nov. 24, 2012

(51) Int. Cl. A61K 36/82; A61K 31/355; A61K 45/06; A61K 31/05

(52) U.S. Cl. CPC A61K 36/82; A61K 31/05; A61K 31/355; A61K 45/06
USPC 424/729

(57) ABSTRACT

Truely Re'Vived is a Natural, holistic product line that allows an alternative choice for a natural way of life. By combining key natural ingredients Truely Re' Vived sets forth the natural healing processes (faster than the normal pace) without the use of chemicals. Truly Re'Vived's has a variety of phenomenal products: “Earth” was created to heal a variety of skin disorders. “Wind” was created to revive, and restore the balance of all grades of hair, giving it life. “Sun” was created to moisturize and heal lip irritancies. “Moon” is a Body Lotion that revitalizes the skins for a healthy appearance. Truely Re'Vived is the alternative to medicine and a true gift that the worlds needs.

### BRIEF DETAIL DESCRIPTION

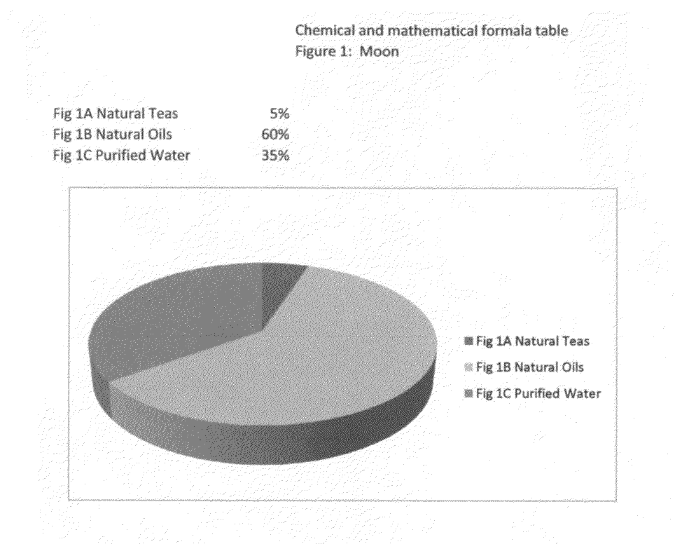

FIG. 1: Truly Re'Vived Moon

The following ingredients combined, creates, Truely Re' Vived's, “Moon” a (Natural Body Lotion):

FIG. 1A: Natural Tea/s and thereof: Natural Tea/s which are high in Polyphenols, Antioxidants that has the potential to contributes to better health.

FIG. 1B: Minerals and oils thereof, with a variety of valuable antioxidants that are not found in other oils. Hydroxytyrosol is thought to be the main antioxidant compound and oil that it exerts anti-inflammatory, antithrombotic, antihypertensive. Tocopherols and tocotrienols, and has many biological functions, the antioxidant function being the most important and/or best known. Vegetable glycerol. It is a carbohydrate that is usually derived from plant oils, and is also used in place of alcohol to extract botanicals, and is an organic compound. Emulsifying wax is a cosmetic emulsifying ingredient.

FIG. 1C: Purified Water. Purification removes contaminants which may interfere with processes, or leave residues upon evaporation. Purified water is water that is mechanically filtered or processed to be cleaned for consumption.

The combining of the ingredients listed above, make up a Body Lotion that revitalizes the skin for a healthy appearance. It's called “MOON”.

Reference: Patent/28146/2

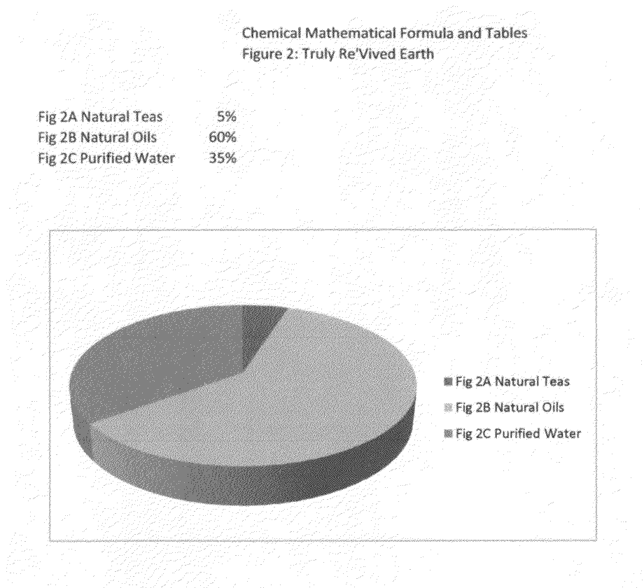

FIG. 2: Truly Re'Vived Earth

The Following ingredients combined create, Truely Re' Vived's, “Earth” (Healing Lotions):

FIG. 2A: Natural Tea/s and thereof: Natural Tea/s which are high in Polyphenols, Antioxidants that has the potential to contributes to better health.

FIG. 2B: Minerals and oils thereof, with a variety of valuable antioxidants that are not found in other oils. Hydroxytyrosol is thought to be the main antioxidant compound. and oil that it exerts anti-inflammatory, antithrombotic, antihypertensive. Tocopherols and tocotrienols, and has many biological functions, the antioxidant function being the most important and/or best known. Vegetable glycerol. It is a carbohydrate that is usually derived from plant oils, and is also used in place of alcohol to extract botanicals, and is an organic compound. Emulsifying wax is a cosmetic emulsifying ingredient.

FIG. 2C: Purified Water. Purification removes contaminants which may interfere with processes, or leave residues upon evaporation. Purified water is water that is mechanically filtered or processed to be cleaned for consumption.

The combining of the ingredients above make up a Healing lotion/cream that heal a variety of skin disorders called “Earth”.

SIMILAR PATENTS: To “EARTH” patents/28146/2/patents/app/20120270835 eczema cream patent

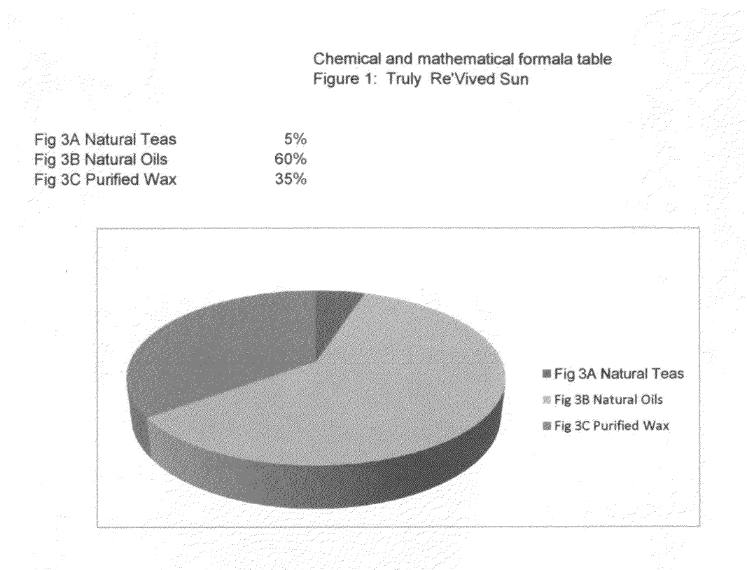

FIG. 3: Truly Re'Vived Sun

The Following ingredients combine creates, Truely Re' Vived's, “Sun”, (Lip Balm):

FIG. 3A: Natural Tea/s and thereof: Natural Tea/s which are high in Polyphenols, Antioxidants that has the potential to contributes to better health.

FIG. 3B: Minerals and a oil thereof, with a valuable antioxidants that are not found in other oils. Hydroxytyrosol is thought to be the main antioxidant compound, and oil that exerts antiinflamatory, antithrombotic, antihypertensive. Tocopherols and tocotrienols,and has many biological functions, the antioxidant function being the most important and/or best known. Vegetable glycerol. It is a carbohydrate that is usually derived from plant oils. It is also used in place of alcohol to extract botanicals, and is an organic compound.

FIG. 3C: Beeswax is a natural wax produced in the bee hive of honey bees, thereof.

The ingredients listed above combined together create the product “SUN”, and it moisturize and heal lip irritancies.

Similar ingredients are being used in other patented products: patents/28146/2/USPTO Application #: #20100178284 Class: 424 946 (USPTO)—Jul. 15, 2010—Class 424 Inventor: Jean-Jacques Debetencourt USPTO Application #: #20120237627—Class: 424769(USPTO)—Sep. 20, 2012—Class 424

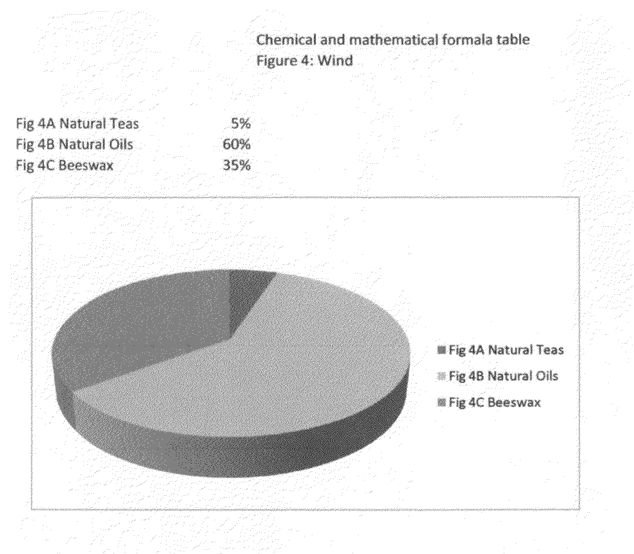

FIG. 4: Truely Re'Vived Wind

The Following ingredients combined create “ Wind” (hair grease,) that was created to revive, and restore

the balance of all grades of hair giving it life:

FIG. 4A: Natural Tea/s and thereof: Natural Tea/s which are high in Polyphenols, Antioxidants that has the potential to contributes to better health.

FIG. 4B: Minerals and a oil thereof, with a variety of valuable antioxidants that are not found in other oils. Hydroxytyrosol is thought to be the main antioxidant compound, and oil that exerts anti-inflammatory, antithrombotic, antihypertensive. Tocopherols and tocotrienols, and has many biological functions, the Antioxidant function being the most important and/or best known. Vegetable glycerol. It is a Carbohydrate that is usually derived from plant oils. It is also used in place of alcohol to extract Botanicals, and is an organic compound.

FIG. 4C: Beeswax is a natural wax produced in the bee hive of honey bees, and thereof.

The ingredients listed above combined together creates a product that revives and restores the balance of all grades of hair, and giving it life, named “WIND”.

Similar ingredients are being used in other patented products:

patents/28146/2/USPTO Application #: #20100178284 Class: 424 946 (USPTO)—Jul. 15, 2010—Class 424 Inventor: Jean-Jacques Debetencourt/USPTO Application #: #20120237627—Class: 424769(USPTO)—Sep. 20, 2012—Class 424

1. Many countries have searched for a natural resolution to resolve issues concerning the skin and hair for many years. Through trial and error and a prayer Truely Re'Vived was born. Each entity of this product line reveals the ability to heal the skin and nurture the hair and scalp. By simply applying the choice product: “Earth” heals a variety of skin disorders, “Wind” was created to revive and restore the balance of all grades of hair, giving it life. “Sun” was created to moisturize and heal lip irritancies. “Moon” is a Body Lotion that revitalizes the skins for a healthy appearance. There is only one alternative Truely Re'Vived created by Seanterel.
